(19) United States
(12) Patent Application Publication
(10) Pub. No.: US 2015/0190270 A1
(43) Pub. Date: Jul. 9, 2015

(54) Sling for Comfortably Supporting a Body Part

(71) Applicant: Martin Boone, East Palo Alto, CA (US)

(72) Inventor: Martin Boone, East Palo Alto, CA (US)

(21) Appl. No.: 14/150,536

(22) Filed: Jan. 8, 2014

(51) Int. Cl. A61F 5/37

(52) U.S. Cl. CPC A61F 5/3738; A61F 5/37

(57) ABSTRACT

The apparatus described is for supporting a body part of a human body including a sleeve, a band, and an attachment mechanism. The sleeve supports the body part minimizing stress on the body part. The band secures the sleeve to stabilize the body part into a static position. The attachment mechanism secures the band to the body such that stress is not incurred by a neck of the body.

### FIELD

The subject matter of this application relates to an apparatus for supporting a human body part. In particular, the subject matter of this application relates to an apparatus that supports a body part, such as a limb including an arm or a leg, for example, and minimizes the stress on the body part to allow the body part to properly heal.

### BACKGROUND

Typically, an apparatus for supporting a body part, such as an arm, currently consists of a sleeve which cradles the arm with a strap, or sling placed over the head and anchored around a portion the neck. Such an apparatus is shown in FIG. 1. However, this apparatus is not advantageous because the weight of the arm is carried by the neck causing severe neck pain and distress. Therefore, there is a need for an apparatus that can support a body part, such as an arm, without anchoring the apparatus around the neck and without requiring the weight of the arm to be carried by the neck.

Additionally, when a person is suffering from a leg injury requiring crutches, as shown in FIG. 2, the person is required to hold their leg suspended above the ground in order to move about on the crutches. For many, this can be a tiresome and labor intensive task creating the potential for exacerbating the injury. Therefore, there is a need for an apparatus to support the leg without requiring the person to hold the leg up based on their own strength.

### SUMMARY OF THE INVENTION

To meet these needs, the present invention includes an example embodiment for an apparatus for supporting a body part of a body. The apparatus includes a sleeve, a band, and an attachment mechanism. The sleeve supports the body part minimizing stress on the body part. The band secures the sleeve to stabilize the body part into a static position. The attachment mechanism secures the band to the body such that stress is not incurred by a neck of the body.

In another example embodiment, an apparatus for supporting an arm of a body includes a sleeve, a band, and an attachment mechanism. The sleeve supports the body part minimizing stress on the arm. The band secures the sleeve to stabilize the arm in a static position. The attachment mechanism secures the band to an upper body garment, which is worn on the body, on at least one shoulder area of the upper body garment.

In yet another example embodiment, an apparatus for supporting an arm of a body includes a sleeve, a band, and an attachment mechanism. The sleeve supports the body part minimizing stress on the arm. The band secures the sleeve to stabilize the arm in a static position. The attachment mechanism secures the band over at least one shoulder of the body and to a lower body garment worn on the body such that stress is not incurred by a neck of the body.

In yet another example embodiment, an apparatus for supporting a leg of a body includes a sleeve, a band, and an attachment mechanism. The sleeve supports the body part minimizing stress on the leg. The band secures the sleeve to stabilize the leg in a static position. The attachment mechanism secures the band to a lower body garment worn on the body such that the leg is not self-suspended by the body.

### BRIEF DESCRIPTION OF THE DRAWINGS

FIG. 1 depicts a prior art sling that requires carrying the weight of an arm around a person's neck.

FIG. 2 depicts a prior art system whereby crutches are used to support a body upright when a leg is injured.

FIG. 3 illustrates an example embodiment of the invention for supporting a body part using a sling that is attached to an upper body garment instead of around a neck.

FIG. 4 illustrates another example embodiment of the invention for supporting a body part using a sling that is attached to a lower body garment instead of around a neck.

FIG. 5 illustrates another example embodiment of the invention for supporting a body part, such as a leg, using a sling that is attached to a lower body garment instead of through self-suspension by the person.

### DETAILED DESCRIPTION OF ONE OR MORE EMBODIMENTS

The disclosure is particularly applicable to the medical and physical therapy fields that deal with stabilizing and supporting an injured limb that has been properly set or positioned during the healing process. It is in this context that the disclosure will be described. However, one of ordinary skill in the art will appreciate that the apparatuses and methods described in accordance with the invention have greater utility, such as for use by outdoor recreation enthusiasts due to the ease in portability of the apparatus or within the military battlefield to quickly stabilize and treat injured limbs.

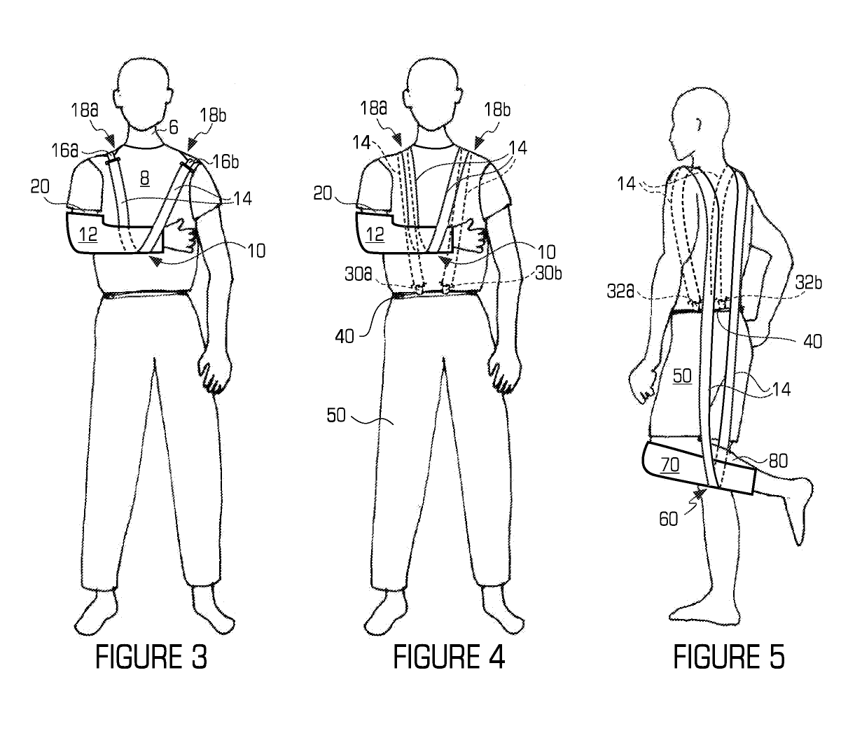

FIG. 3 illustrates an embodiment of the invention for supporting a body part using a sling that is attached to an upper body garment 8 instead of around a neck 6. The apparatus 10 includes a sleeve 12, a band 14, and attachment mechanisms 16a, 16b. The sleeve 12 serves the purpose of minimizing stress to a body part, such as an arm 20, and keeping the body part rigid. For example, the sleeve 12 may be a cast or a splint that is routinely used for stabilizing and holding a broken limb into the position at which the limb was set in order for the limb to heal properly. The sleeve 12 is placed in a band 14, which is attached to an upper body garment 8 by attachment mechanisms 16a, 16b. The band 14 supports the sleeve 12 so that the arm 20 is stabilized into a static position. The attachment mechanisms 16a, 16b may be placed on shoulder areas 18a, 18b, respectively, so that the sleeve 12 is suspended by the band 14 supporting the arm 20, for example.

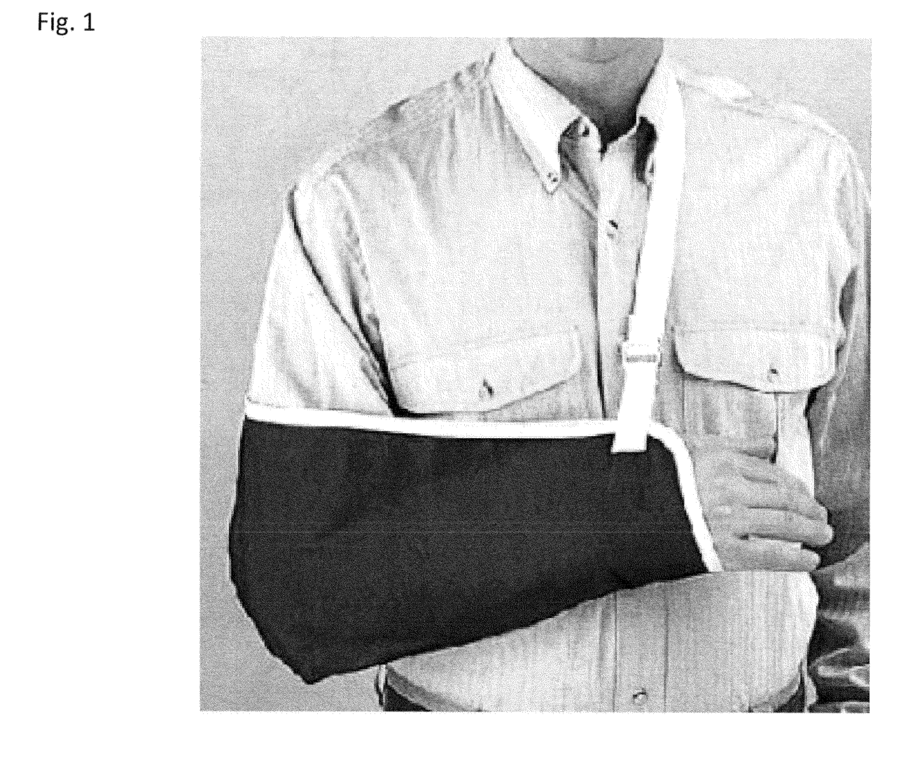

Since the attachment mechanisms 16a, 16b are placed on the shoulder areas 18a, 18b, a strap around the neck to support the arm 20 is not required. This prevents strain to the neck and also prevents neck pain that would have resulted from the prior art apparatus of FIG. 1. Further, the weight of the arm 20 is not carried by one point, i.e., around the neck, and is instead distributed across two points, the shoulders, in the present embodiment. This novel arrangement provides for a more stabilized weight distribution without causing pain or strain to the neck.

FIG. 4 illustrates an embodiment of the invention for supporting a body part using a sling that is attached to a lower body garment 50 instead of around a neck 6. The apparatus 10 includes a sleeve 12, a band 14, and attachment mechanisms 30a, 30b. The sleeve 12 serves the purpose of minimizing stress to a body part, such as an arm 20, and keeping the body part rigid. The sleeve 12 is placed in a band 14, which is attached to a lower body garment 50 by attachment mechanisms 30a, 30b. The band 14 supports the sleeve 12 so that the arm 20 is stabilized into a static position. The attachment mechanisms 30a, 30b may be placed on a waistband portion 40 of the lower body garment 50, so that the sleeve 12 is suspended by the band 14 supporting the arm 20, for example.

Since the band 14 goes over the shoulder areas 18a and 18b and the attachment mechanisms 30a, 30b may be placed on the waistband 40, a strap around the neck to support the arm 20 is not required. This prevents strain to the neck and also prevents neck pain that would have resulted from the prior art apparatus of FIG. 1. Further, the weight of the arm 20 is not carried by one point, i.e., around the neck, and is instead distributed across two points, the shoulders, in the present embodiment. This arrangement provides for a more stabilized weight distribution without causing pain or strain to the neck.

FIG. 5 illustrates an embodiment of the invention for supporting a body part using a sling that is attached to a lower body garment 50 instead of through self-suspension by the person. The apparatus 60 includes a leg sleeve 70, a band 14, and attachment mechanisms 32a, 32b. The leg sleeve 70 serves the purpose of minimizing stress to a body part, such as a leg 80, and keeping the body part rigid. The leg sleeve 70 is placed in a band 14, which is attached to a lower body garment 50 by attachment mechanisms 32a, 32b similar to suspender clips. The band 14 supports the leg sleeve 70 so that the leg 80 is stabilized into a static position. The attachment mechanisms 32a, 32b may be placed on a waistband portion 40 of the lower body garment 50, so that the leg sleeve 70 is suspended by the band 14 supporting the leg 80, for example.

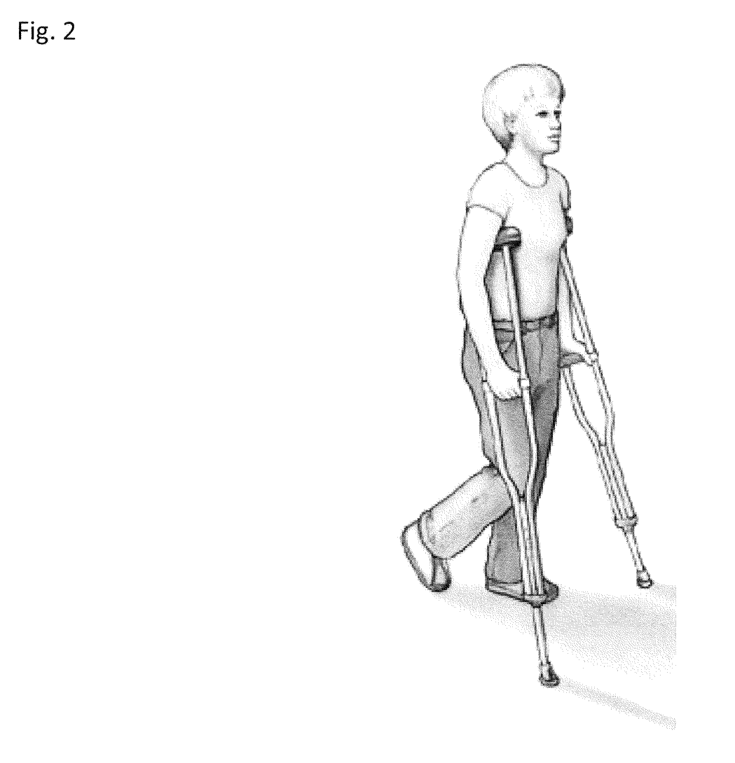

Since the attachment mechanisms 32a, 32b may be placed on the waistband 40 and the band 14 supports the leg sleeve 70, self-suspension of the leg by the person is not required. This prevents strain to the person that would have resulted from the prior art apparatus of FIG. 2. This novel arrangement provides for a more stabilized weight distribution and allows the person to focus on balance with the crutches without having to also focus on holding up the injured limb.

Across any embodiment, the sleeve 12 may be a cast, a conventional splint, or a splint made of material that can be rolled around the limb and filled with air to stabilize the limb and hold the limb in a rigid state. One of ordinary skill in the art will appreciate that this apparatus can be combined with any device that stabilizes and keeps the limb static or rigid.

Further, the band 14 is adjustable in length so that any band can accommodate the varying sizes of people including their arm and leg length and height. The means to adjust the band may include a roller type device or a tri-bar strap adjuster attached to the band 14.

In addition, the attachment mechanisms 18a, 18b, 30a, 30b, 32a, 32b may be any clip that is able to be placed at the end of a strap, such as band 14 and able to clip or attach the band 14 to a garment worn by a person. The attachment mechanisms 18a, 18b, 30a, 30b, 32a, 32b can also be velcro, S hooks or a hole and button arrangement. One of ordinary skill in the art will appreciate other attachment arrangements that are able to attach the band 14 to a garment.

While the foregoing has been with reference to a particular embodiment of the invention, it will be appreciated by those skilled in the art that changes in this embodiment may be made without departing from the principles and spirit of the disclosure, the scope of which is defined by the appended claims.

What is claimed is:

1. An apparatus for supporting a body part of a human body, comprising:
a sleeve for supporting the body part;
a band for securing the sleeve to stabilize the body part into a static position; and
an attachment mechanism for securing the band to a garment on the body in at least two locations.

2. The apparatus of claim 1, wherein the sleeve is rigid and wraps around the body part.

3. The apparatus of claim 2, wherein the sleeve is filled with air and wraps around the body part.

4. The apparatus of claim 1, wherein the band is adjustable in length.

5. The apparatus of claim 2, wherein the band is adjustable in length.

6. The apparatus of claim 3, wherein the band is adjustable in length.

7. The apparatus of claim 1, wherein the body part is at least one of an arm and a leg.

8. The apparatus of claim 1, wherein the two locations are near the shoulders of the body.

9. The apparatus of claim 3, wherein the two locations are near the shoulders of the body.

10. An apparatus for supporting an arm of a human body, comprising:
a sleeve for supporting the arm;
a band for securing the sleeve to stabilize the arm in a static position; and
at least one attachment mechanism for securing the band to a garment worn on the body.

11. The apparatus of claim 10, wherein the garment is an upper body garment.

12. The apparatus of claim 10, wherein the garment is a lower body garment.

13. The apparatus of claim 10, wherein the sleeve is rigid and wraps around the arm.

14. The apparatus of claim 13, wherein the sleeve is filled with air and wraps around the arm.

15. The apparatus of claim 10, wherein the band is adjustable in length.

16. An apparatus for supporting a leg of a human body, comprising:
a sleeve for supporting the leg;
a band for securing the sleeve to stabilize the leg in a static position; and
at least one attachment mechanism for securing the band to a lower body garment worn on the body.

17. The apparatus of claim 16, wherein the at least one attachment mechanism is attached to a waistband portion of the lower body garment.

18. The apparatus of claim 16, wherein the sleeve is rigid and wraps around the leg.

19. The apparatus of claim 18, wherein the sleeve is filled with air and wraps around the leg.

20. The apparatus of claim 16, wherein the band is adjustable in length.
